(19) United States
(12) Patent Application Publication
(10) Pub. No.: US 2006/0210507 A1
(43) Pub. Date: Sep. 21, 2006

(54) Cosmetic composition comprising at least one sulphonic polyester and at least one polyurethane

(76) Inventors: Ludivine Laurent, Courbevoie (FR); Katarina Benabdillah, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/260,440

(22) Filed: Oct. 28, 2005

Related U.S. Application Data
(60) Provisional application No. 60/646,495, filed on Jan. 25, 2005.

(30) Foreign Application Priority Data
Oct. 28, 2004 (FR) 04 52471

(51) Int. Cl. A61K 8/87

(52) U.S. Cl. 424/70.7; 424/70.17

(57) ABSTRACT

Disclosed herein is a cosmetic composition comprising, in a cosmetically acceptable medium, at least one water-dispersible sulphonic polyester and at least one high molecular weight polyurethane. Also disclosed herein is a method for shaping, fixing, and/or holding the hair comprising applying this cosmetic composition to the hair.

This application claims benefit of U.S. Provisional Application No. 60/646,495, filed January 25, 2005, the contents of which are incorporated herein by reference. This application also claims benefit of priority under 35 U.S.C. § 119 to French Patent Application No. 04 52471, filed Oct. 28, 2004, the contents of which are also incorporated by reference.

Disclosed herein is a cosmetic composition comprising, in a cosmetically acceptable medium, at least one water-dispersible sulphonic polyester and at least one high molecular weight polyurethane. Also disclosed herein is a method of shaping and/or holding a hairstyle comprising applying this cosmetic composition to the hair.

As used herein, the term “high molecular weight polyurethane” means a polyurethane having a number-average molecular weight ranging from 400 000 to 5 000 000 g/mol inclusive as determined by gel permeation chromatography (GPC).

Cosmetic compositions for shaping and/or holding the hairstyle may be spray compositions comprising a solution, which is usually alcoholic, and at least one component known as a fixative component, which is generally a polymeric resin and whose function is to form weld points between the individual hairs or to coat them. These fixative components are often formulated as a blend with various cosmetic adjuvants.

These cosmetic compositions are generally presented either in a pump-dispenser bottle or in a suitable aerosol container pressurized by means of a propellant; the aerosol system containing a liquid phase (or fluid) and a propellant. It is also possible to use styling compositions in the form of gels, creams, or mousses.

Once applied to the hair, the fixative compounds should allow the hair to be fixed.

However, the polymers presently in use as fixatives in styling compositions do not allow the shape of the hairstyle to be retained when it comes into contact with liquid water for a prolonged period, such as, for example, when contacted with rain, perspiration, or during bathing, such as bathing in the sea, in a swimming pool, etc.

The present inventors have discovered that the use of a cosmetic composition comprising, in a cosmetically acceptable medium, at least one water-dispersible sulphonic polyester and at least one high molecular weight polyurethane may allow hairstyles to be fixed and shaped and the shape of the hairstyles to be retained when the hair comes into contact with liquid water for a prolonged period. For the purposes of the present disclosure, this phenomenon is referred to as “water resistance”.

As used herein, the term “prolonged period” means contact with water for a minimum period of one minute, for example, 10 minutes, or 20 minutes.

The compositions of the present disclosure also make it possible to obtain a hairstyle resistant to atmospheric humidity.

The compositions according to the present disclosure may allow effective fixation and effective holding of the hair, i.e., a styling effect that lasts throughout the day, or even for several days, and which exhibits good water resistance, such as resistance to repeated bathing. These compositions may also exhibit the additional advantage of being removed by shampooing. These compositions may also make it possible to impart good cosmetic properties.

One embodiment of the present disclosure provides a cosmetic composition comprising, in a cosmetically acceptable medium, at least one water-dispersible sulphonic polyester and at least one polyurethane having a number-average molecular weight ranging from 400 000 to 5 000 000 g/mol.

Another embodiment of the present disclosure provides a method of shaping and/or holding the hairstyle, comprising applying the cosmetic composition according to the present disclosure to the hair.

A further embodiment of the present disclosure provides a method for fixing and/or holding the hair, for example, when the hair is contacted with liquid water for a prolonged period of time, i.e., in the case of rain, perspiration, and/or bathing, such as bathing in the sea or in a swimming pool, the method comprising applying a cosmetic styling composition to the hair, wherein the cosmetic styling composition comprises, in a cosmetically acceptable medium, at least one water-dispersible sulphonic polyester and at least one polyurethane having a number-average molecular weight ranging from 400 000 to 5 000 000 g/mol.

According to these embodiments, the use of the cosmetic composition disclosed herein allows a water-resistant hairstyle (shaped hair) to be obtained.

The cosmetic composition according to the present disclosure may be in any form, for example, lotions, sprays, mousses, gels, creams, etc.

As used herein, the term “cosmetic styling composition” means a composition for shaping and/or holding the hairstyle.

Other subjects, features, aspects, and advantages of the disclosure will emerge even more clearly upon reading the description and the examples which follow.

Cosmetically Acceptable Medium

The cosmetically acceptable medium used in the compositions according to the present disclosure may be an aqueous medium or a mixture of water and at least one organic solvent.

The at least one organic solvent used in the compositions according to the present disclosure may be C₁-C₄ lower alcohols such as ethanol, isopropanol, tert-butanol, and n-butanol; polyols; and polyol ethers. In at least one embodiment, the solvent may be chosen from ethanol, propylene glycol, and glycerol.

The at least one solvent may be present in the composition in an amount ranging from 0 to 70%, for example, from 0 to 50%, or from 0 to 15% by weight relative to the total weight of the composition.

In one embodiment, the composition of the present disclosure may be free of C₁-C₄ alcohols.

Additional organic solvents useful in the compositions according to the present disclosure include, for example, polyols such as propylene glycol, polyol ethers, and mixtures thereof.

The additional organic solvent(s) may be present in the composition in an amount ranging from 0 to 70%, for example, from 0 and 50%, or from 0 to 15% by weight relative to the total weight of the composition.

Sulphonic Polyester

The composition according to the present disclosure comprises at least one water-dispersible sulphonic polyester. The sulphonic polyester may be linear or branched.

As used herein, a “water-dispersible sulphonic polyester” is any sulphonic polyester capable of forming a dispersion, which is a two-phase system in which the first phase is in the form of finely divided particles distributed uniformly in the second phase, which is the continuous phase.

As used herein, the term “sulphonic polyester” means a copolyester obtained by polycondensation of at least one dicarboxylic acid or one of its esters, at least one diol, and at least one sulphoaryidicarboxylic difunctional compound substituted on the aromatic nucleus by a group —SO₃M, in which M is chosen from hydrogen and metal ions such as Na⁺, Li⁺, and K⁺.

The at least one water-dispersible linear sulphonic polyester in accordance with at least one embodiment of the present disclosure may have a weight-average molecular mass ranging from 1000 to 60 000, for example, from 4000 to 20 000, as determined by gel permeation chromatography (or GPC).

The glass transition temperature (Tg) of the at least one linear sulphonic polyester may range from 10° C to 100° C. In at least one embodiment, the Tg of the at least one polyester may be greater than or equal to 50° C.

The glass transition temperature (Tg) may be measured by differential enthalpy analysis (DSC, differential scanning calorimetry) in accordance with ASTM D3418-97.

Suitable sulphonic polyesters may include, for example, those described in U.S. Pat. Nos. 3,734,874, 3,779,993, 4,119,680, 4,300,580, 4,973,656, 5,660,816, 5,662,893, and 5,674,479.

In at least one embodiment of the present disclosure, the at least one sulphonic polyester may include at least units derived from isophthalic acid, from a sulphoaryldicarboxylic salt, and from diethylene glycol. For example, the at least one sulphonic polyester may be obtained from isophthalic acid, the sodium salt of sulphoisophthalic acid, diethylene glycol, and 1,4-cyclohexanedimethanol.

Examples of linear sulphonic polyesters include, for example, those known under the INCI name Diglycol/CHDM/Isophthalates/SIP and sold under the trade names Eastman AQ polymer (AQ35S, AQ38S, AQ55S, and AQ48 Ultra) by Eastman Chemical.

Examples of branched sulphonic polyesters include, but are not limited to, those described in European Patent No. 1 083 867, such as AQ 1350, sold by Eastman Chemical.

The at least one water-dispersible sulphonic polyester may be present in the composition in an amount ranging from 0.1% to 40%, for example, from 1% to 30%, or from 5% to 25% by weight relative to the total weight of the composition.

Polyurethanes

The composition according to the present disclosure comprises at least one high-molecular weight polyurethane having a number-average molecular weight ranging from 400 000 to 5 000 000 g/mol.

As used herein, the term “polyurethane” means a polycondensate comprising at least one polyurethane sequence. Such polycondensates are described, for example, in European Patent Nos. 0 751 162, 0 637 600, 0 648 485, 0 656 021, and 0 619 111, French Patent No. 2 743 297, and International Publication No. WO 94/03510.

The at least one polyurethane used in accordance with the present disclosure may be soluble in the cosmetically acceptable aqueous medium, for example, after neutralization with an organic or inorganic base, or may form a dispersion in said medium. In that case, the dispersion may comprise at least 0.05% by weight of at least one surfactant, allowing the non-associative polyurethane to be dispersed and to be held in dispersion. According to at least one embodiment, the at least one surfactant used in the dispersion may be any type of surfactant, for example, a nonionic surfactant.

The average size of the particles of the non-associative polyurethane in the dispersion may range from 0.1 to 1 micrometer.

In at least one embodiment of the present disclosure, the polyurethane may be formed by an arrangement of blocks, this arrangement being obtained, for example, from:
(1) at least one compound containing two or more active hydrogen atoms per molecule;
(2) at least one diol or a mixture of diols containing at least one acidic function, and their salts; and
(3) at least one di- or polyisocyanate.

The compound (1) may, in certain embodiments, be chosen from diols, diamines, polyesterols, polyetherols, and mixtures thereof.

In at least one embodiment, the compound (1) may be chosen from linear polyethylene glycols and polypropylene glycols, for example, those obtained by reacting ethylene oxide or propylene oxide with water or reacting diethylene or dipropylene glycol in the presence of sodium hydroxide as catalyst. These polyalkylene glycols may have a molecular mass ranging from 600 to 20 000.

Other suitable organic compounds include those comprising at least one group chosen from mercapto, amino, carboxyl, and hydroxyl groups. These compounds include, for example, polyhydroxy compounds such as polyether-diols, polyester-diols, polyacetal-diols, polyamide-diols, polyester-polyamide-diols, poly(alkylene ether)-diols, polythioether-diols, and polycarbonate-diols.

Examples of polyether-diols include, but are not limited to, condensation products of ethylene oxide, propylene oxide, or tetrahydrofuran, their copolymerization or condensation products, grafted or in blocks, such as mixtures of condensates of ethylene oxide and propylene oxide, and the products of high-pressure polymerization of olefins with alkylene oxide condensates. Suitable polyether-diols may be prepared, for example, by condensing alkylene oxides and polyhydric alcohols, such as ethylene glycol, propylene 1,2-glycol, and 1,4-butanediol.

The polyester-diols, polyester-amides, and polyamide-diols may be saturated and may be obtained, for example, from the reaction of saturated or unsaturated polycarboxylic acids with polyhydric alcohols, diamines, and/or polyamines. These compounds may be prepared using, for example, adipic acid, succinic acid, phthalic acid, terephthalic acid, and maleic acid. Polyhydric alcohols appropriate for preparing the at least one polyester include, for example, ethylene glycol, propylene 1,2-glycol, 1,4-butanediol, neopentyl glycol, and hexanediol. It is also possible to use amino alcohols, for example, ethanolamine. Appropriate diamines for preparing the amide-polyesters include, for example, ethylenediamine and hexamethylenediamine.

Suitable polyacetals may be prepared, for example, from 1,4-butanediol, hexanediol, and formaldehyde. Suitable polythioethers may be prepared, for example, by a condensation reaction of thioglycols, alone or in combination with other glycols such as ethylene glycol and propylene 1,2-glycol or with other polyhydroxy compounds. Polyhydroxy compounds already containing urethane groups, natural polyols, which may additionally be modified, for example, castor oil and carbohydrates, may also be used.

In at least one embodiment, the compound of group (1) may be a polyesterol, for example, a polyester-diol formed by the reaction of at least one (di)-polyol (1ₐ) and at least one acid (1_{b}). The (di)-polyol (1ₐ) may be chosen, for example, from neopentyl glycol, 1,4-butanediol, hexanediol, ethylene glycol, diethylene glycol, propylene glycol, butylene glycol, neopentyl glycol, and (di)-polyethylene glycol. The acid (1_{b}) may be chosen, for example, from phthalic acid, isophthalic acid, adipic acid, and (poly)lactic acid.

Compound (2) may be chosen from, for example, hydroxy carboxylic acids, such as dimethylolpropanoic acid (DMPA), and 2,2-hydroxymethyl-carboxylic acids. In at least one embodiment, compound (2) may be useful as a coupling block. In another embodiment, compound (2) may include at least one poly(β,β-dihydroxy carboxylic acid).

Examples of compound (2) include, but are not limited to, 2,2-di(hydroxymethyl)acetic acid, 2,2-dihydroxymethylpropionic acid, 2,2-dihydroxymethylbutyric butyric acid and 2,2-dihydroxymethylpentanoic acid.

The di- or polyisocyanate (3) may be chosen from, for example, hexamethylene diisocyanate, isophorone diisocyanate (IPDI), tolylene diisocyanate, diphenylmethane 4,4′-diisocyanate (DPMD), dicyclohexylmethane 4,4′-diisocyanate (DCMD), methylenedi-p-phenyl diisocyanate, methylenebis(4-cyclohexyl isocyanate), toluene diisocyanates, naphthalene 1,5-diisocyanate, diphenylmethane 4,4′-diisocyanate, 2,2′-dimethyldiphenylmethane 4,4′-diisocyanate, phenylene 1,3-diisocyanate, phenylene 1,4-diisocyanate, mixtures of toluene 2,4- and 2,6-diisocyanates, 2,2′-dichloro-4,4′-diisocyanatodiphenylmethane, 2,4-dibromo-1,5-diisocyanatonaphthalene, butane 1,4-diisocyanate, hexane 1,6-diisocyanate, and cyclohexane 1,4-diisocyanate.

The at least one non-associative polyurethane may be formed with the aid of at least one additional compound (4), serving generally to extend its chain. Compound (4) may be chosen from saturated or unsaturated glycols such as ethylene glycol, diethylene glycol, neopentyl glycol, and triethylene glycol; amino alcohols such as ethanolamine, propanolamine, and butanolamine; heterocyclic, aromatic, cycloaliphatic, and aliphatic primary amines; diamines; carboxylic acids such as aliphatic, aromatic, and heterocyclic carboxylic acids, for instance oxalic, succinic, glutaric, adipic, sebacic, and terephthalic acids; and aminocarboxylic acids. In one embodiment, compound (4) may be chosen from aliphatic diols.

The polyurethanes used according to the present disclosure may also be formed from additional compounds (5) comprising a silicone skeleton, such as polysiloxanes, polyalkylsiloxanes, and polyarylsiloxanes, for example, polyethylsiloxanes, polymethylsiloxanes, and polyphenylsiloxanes, optionally comprising hydrocarbon chains grafted on the silicon atoms.

In at least one embodiment of the present disclosure, the at least one polyurethane may comprise at least one base repeating unit of formula (I):
—O—B—O—CO—NH—R—NH—CO— (I)
in which:

—B is chosen from C₁-C₃₀ divalent hydrocarbon groups, which are unsubstituted or substituted by a group comprising at least one carboxylic acid function and/or at least one sulphonic acid function, said carboxylic and/or sulphonic acid functions being in free form or else fully or partially neutralized by an inorganic or organic base, and

—R is a divalent group chosen from C₁-C₂₀ aliphatic hydrocarbons, C₃-C₂₀ cycloaliphatic hydrocarbons, and C₆-C₂₀ aromatic hydrocarbon groups, for example, C₁-C₂₀ alkylene groups, C₆-C₂₀ arylene groups, C₃-C₂₀ cycloalkylene groups, and combinations thereof, these groups being substituted or unsubstituted.

In at least one embodiment, the group R may be chosen from groups of the following formulae: 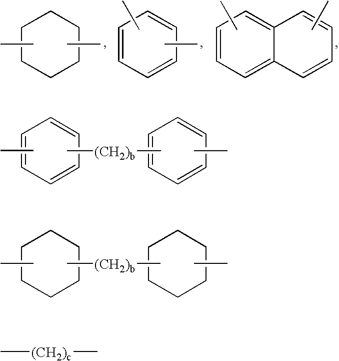
in which b is an integer ranging from 0 to 3, and c is an integer ranging from 1 to 20, for example, from 2 to 12.

For example, the group R may be chosen from hexamethylene, 4,4′-diphenylenemethane, 2,4- and/or 2,6-tolylene, 1,5-naphthylene, p-phenylene, methylene-4,4-biscyclohexyl, and the divalent group derived from isophorone.

The at least one polyurethane used in accordance with the present disclosure may further comprise at least one polysiloxane sequence.

In at least one embodiment of the present disclosure, the polyurethanes described herein may be non-associative, i.e., their structure does not include an alkyl or alkenyl chain containing more than 10 carbon atoms.

The at least one polyurethane may be present in the composition in an amount ranging from 0.05% to 40% by weight, for example, from 0.1% to 20% by weight, or from 1% to 8% by weight relative to the total weight of the composition, for cosmetic treatment of keratin materials.

The at least one polyurethane may, in one embodiment, have a molecular mass ranging from 400 000 to 3 000 000, for example, from 1 000 000 to 2 500 000.

A non-limiting example of a suitable polyurethane is AVALURE UR-450, sold by Noveon, which is an anionic copolymer formed from PPG-17 (polypropylene glycol with n number of units, where n=17)/IPDI (isophorone diisocyanate)/DMPA (dimethylolpropionic acid). In an aqueous medium it is in the form of a dispersion. Its molecular weight is 1 830 000 g/mol.

Optional Additives

The compositions according to the present disclosure may further comprise at least one additional cosmetic adjuvant such as those mentioned below.

Fixative Polymers

The composition disclosed herein may further comprise at least one fixative polymer used in the art, for example, chosen from anionic, cationic, amphoteric, and nonionic fixative polymers, and mixtures thereof.

The fixative polymers may be soluble in the cosmetically acceptable medium or insoluble in said medium, and in that case, may be used in the form of dispersions of solid or liquid polymer particles (latex or pseudolatex).

Suitable anionic fixative polymers include, but are not limited to, polymers comprising groups derived from carboxylic, sulphonic, and/or phosphoric acid, and having a number-average molecular mass ranging from 500 to 5 000 000.

The carboxylic groups may be provided by unsaturated mono- or dicarboxylic acid monomers, such as those of formula (II): 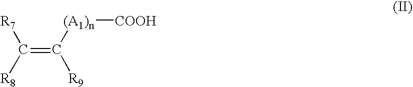
in which:

n is an integer ranging from 0 to 10,

A₁ is a methylene group optionally joined to the carbon atom of the unsaturated group or to the adjacent methylene group when n is greater than 1, via a heteroatom such as oxygen and sulphur,

R₇ is chosen from hydrogen, phenyl, and benzyl,

R₈ is chosen from hydrogen, lower alkyl groups, and lower carboxyl groups,

and

R₉ is chosen from hydrogen, lower alkyl groups, —CH₂—COOH, phenyl, and benzyl.

In formula (II), a lower alkyl group may be a group comprising from 1 to 4 carbon atoms, for example, methyl and ethyl groups.

The anionic fixative polymers may be chosen from homopolymers and copolymers of acrylic and methacrylic acid or their salts, copolymers of crotonic acid, copolymers of C₄-C₈ monounsaturated carboxylic acids or anhydrides, polyacrylamides comprising carboxylate groups, homopolymers and copolymers comprising sulphonic groups, anionic polyurethanes, and grafted anionic silicone polymers.

Examples of anionic fixative polymers comprising carboxylic groups include, but are not limited to:

A) acrylic or methacrylic acid homo- or copolymers or salts thereof, for example, the products sold under the names Versicol® E and K by Allied Colloid and Ultrahold® by BASF, the copolymers of acrylic acid and of acrylamide sold in the form of their sodium salts under the names RETEN 421, 423, and 425 by Hercules, and the sodium salts of polyhydroxycarboxylic acids;

B) copolymers of acrylic or methacrylic acid with a monoethylenic monomer such as ethylene, styrene, vinyl esters, acrylic acid esters, and methacrylic acid esters, optionally grafted onto a polyalkylene glycol such as polyethylene glycol, and optionally crosslinked. Such polymers are described, for example, in French Patent No.1 222 944 and German Patent Application No. 2 330 956; the copolymers of this type comprising an optionally N-alkylated and/or hydroxyalkylated acrylamide unit in their chain as described, for example, in Luxembourg Patent Application Nos. 75370 and 75371 and sold under the name QUADRAMER by American Cyanamid. Other suitable copolymers include copolymers of acrylic acid and of C₁-C₄ alkyl methacrylate and vinylpyrrolidone terpolymers, of acrylic acid and of C₁-C₂₀ alkyl methacrylates, for example of lauryl, such as the product sold by ISP under the name Acrylidone® LM, and methacrylic acid/ethyl acrylate/tert-butyl acrylate terpolymers such as the product sold under the name Luvimer® 100 P by BASF;

These copolymers may further include methacrylic acid/acrylic acid/ethyl acrylate/methyl methacrylate copolymers as an aqueous dispersion, sold under the name Amerhold® DR 25 by Amerchol;

C) crotonic acid copolymers, such as those comprising vinyl acetate or propionate units in their chain and optionally other monomers such as allylic esters and methallylic esters, vinyl ethers and vinyl esters of a linear or branched saturated carboxylic acids with a long hydrocarbon chain, such as those containing at least 5 carbon atoms, these polymers optionally being grafted or crosslinked, and vinyl, allylic, and methallylic ester monomers of an α- or β-cyclic carboxylic acid. Such polymers are described, for example, in French Patent Nos. 1 222 944, 1 580 545, 2 265 782, 2 265 781, 1 564 110, and 2 439 798. Examples of commercial products falling into this class include the resins 28-29-30, 26-13-14 and 28-13-10 sold by National Starch;

D) copolymers of C₄-C₈ monounsaturated carboxylic acids or anhydrides chosen from:
copolymers comprising (i) at least one unit chosen from maleic, fumaric, and itaconic acids and anhydrides and (ii) at least one monomer chosen from vinyl esters, vinyl ethers, vinyl halides, phenylvinyl derivatives, acrylic acid, and acrylic acid esters, the anhydride functions of these copolymers optionally being monoesterified or monoamidated. Such polymers are described, for example, in U.S. Pat. Nos. 2,047,398, and 2,723,248, and British Patent No. 839 805. Examples of commercial products include, but are not limited to, those sold under the names Gantrez® AN and ES by ISP;
copolymers comprising (i) at least one unit chosen from maleic, citraconic, and itaconic anhydride units and (ii) at least one monomer chosen from allylic and methallylic esters optionally comprising at least one group chosen from acrylamide, methacrylamide, α-olefin, acrylic ester, methacrylic ester, acrylic acid, methacrylic acid, and vinylpyrrolidone groups in their chain,

wherein the anhydride functions of these copolymers are optionally monoesterified or monoamidated. These polymers are described, for example, in French Patent Nos. 2 350 384 and 2 357 241; and

E) polyacrylamides comprising carboxylate groups.

Homopolymers and copolymers comprising sulphonic groups include, but are not limited to, polymers comprising vinylsulphonic, styrenesulphonic, naphthalenesulphonic, and acrylamidoalkylsulphonic units. These polymers may be selected in particular from:
polyvinylsulphonic acid salts having a molecular mass ranging from 1000 to 100 000, and also copolymers comprising at least one unsaturated comonomer such as acrylic and methacrylic acids and their esters, acrylamide and its derivatives, vinyl ethers, and vinylpyrrolidone;
polystyrenesulphonic acid salts such as the sodium salts sold, for example, under the names Flexan® 500 and Flexan® 130 by National Starch. These compounds are described, for example, in French Patent No. 2 198 719;
polyacrylamidesulphonic acid salts, such as those described in U.S. Pat. No. 4,128,631, for example, polyacrylamidoethylpropanesulphonic acid, sold under the name COSMEDIA POLYMER HSP 1180 by Henkel.

Another non-limiting example of a suitable anionic fixative polymer is the branched block anionic polymer sold under the name FIXATE G-100 by Noveon.

According to at least one embodiment of the present disclosure, the anionic fixative polymers may be chosen from acrylic acid copolymers, such as the acrylic acid/ethyl acrylate/N-tert-butylacrylamide terpolymers sold, for example, under the name Ultrahold® Strong by BASF; copolymers derived from crotonic acid, such as vinyl acetate/vinyl tert-butylbenzoate/crotonic acid terpolymers and the crotonic acid/vinyl acetate/vinyl neododecanoate terpolymers sold, for example, under the name RÉSINE 28-29-30 by National Starch; polymers derived from maleic, fumaric, and itaconic acids and anhydrides with vinyl esters, vinyl ethers, vinyl halides, phenylvinyl derivatives, acrylic acid, and acrylic acid esters, such as the methyl vinyl ether/monoesterified maleic anhydride copolymers sold, for example, under the name Gantrez® by ISP; the copolymers of methacrylic acid and of methyl methacrylate sold under the name Eudragit® L by Rohm Pharma; the copolymers of methacrylic acid and of ethyl acrylate sold under the names Luvimer® MAEX and MAE by BASF; the vinyl acetate/crotonic acid copolymers sold under the name LUVISET CA 66 by BASF; the vinyl acetate/crotonic-acid copolymers grafted with polyethylene glycol sold under the name Aristoflex® A by BASF; and the polymer sold under the name FIXATE G-100 by Noveon.

In at least one embodiment, the anionic fixative polymers may be chosen from the methyl vinyl ether/monoesterified maleic anhydride copolymers sold under the name Gantrez® ES 425 by ISP; the acrylic acid/ethyl acrylate/N-tert-butylacrylamide terpolymers sold under the name Ultrahold® Strong by BASF; the copolymers of methacrylic acid and of methyl methacrylate sold under the name Eudragit® L by Rohm Pharma; the vinyl acetate/vinyl tert-butylbenzoate/crotonic acid terpolymers and the crotonic acid/vinyl acetate/vinyl neododecanoate terpolymers sold under the name RÉSINE 28-29-30 by National Starch; the copolymers of methacrylic acid and of ethyl acrylate sold under the names Luvimer® MAEX and MAE by BASF; the vinylpyrrolidone/acrylic acid/lauryl methacrylate terpolymers sold under the name Acrylidone® LM by ISP; and the polymer sold under the name FIXATE G-100 by Noveon.

Cationic fixative film-forming polymers that may be used according to the present disclosure may be chosen from polymers comprising primary, secondary, tertiary, and/or quaternary amine groups forming part of the polymer chain or directly attached thereto, and having a molecular weight ranging from 500 to 5 000 000, for example, from 1000 to 3 000 000.

In one embodiment of the present disclosure, the cationic fixative polymers may be chosen from homopolymers and copolymers of acrylic and methacrylic esters and amides comprising amine functions, cationic polysaccharides, quaternary vinylpyrrolidonevinylimidazole copolymers, and chitosans.

These cationic polymers may be chosen from, for example:

(1) homopolymers and copolymers derived from acrylic esters, methacrylic esters, and amides and comprising at least one unit chosen from units of formulae (A)-(C): 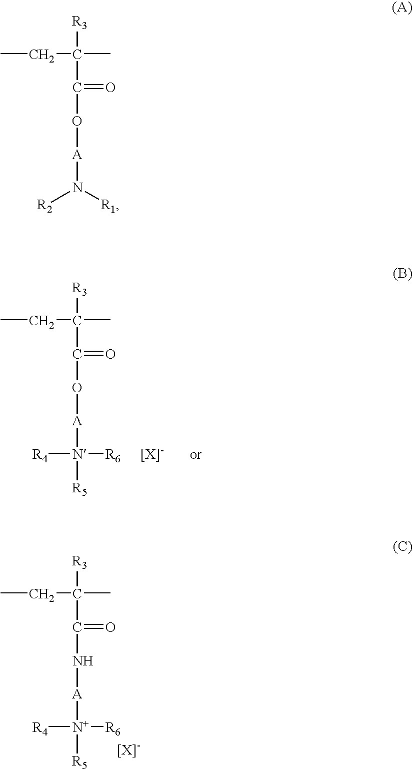
in which:

R₃ is chosen from hydrogen and CH₃;

A is chosen from linear or branched alkyl groups comprising from 1 to 6 carbon atoms and hydroxyalkyl groups comprising from 1 to 4 carbon atoms;

R₄, R₅, and R₆, which may be identical or different, are chosen from alkyl groups comprising from 1 to 18 carbon atoms and from benzyl radicals;

R₁ and R₂, which may be identical or different, are chosen from hydrogen and alkyl groups comprising from 1 to 6 carbon atoms;

X is chosen from methosulphate anions and halides such as chloride and bromide.

The copolymers of class (1) may also contain at least one comonomer unit chosen from acrylamides, methacrylamides, diacetone acrylamides, acrylamides, and methacrylamides substituted on the nitrogen by lower (C₁-C₄) alkyl groups; groups derived from acrylic acid, methacrylic acid, acrylic acid esters, and methacrylic acid esters; vinyllactams such as vinylpyrrolidone and vinylcaprolactam; and vinyl esters.

Examples of copolymers of class (1), include, but are not limited to:

copolymers of acrylamide and of dimethylaminoethyl methacrylate quaternized with dimethyl sulphate or with a dimethyl halide, such as the product sold under the name Hercofloc® by Hercules,

copolymers of acrylamide and of methacryloyloxyethyltrimethylammonium chloride, described, for example, in European Patent Application No. 0 080 976 and sold under the name BINA QUAT P 100 by Ciba Geigy,

copolymers of acrylamide and of methacryloyloxyethyltrimethylammonium methosulphate, such as the product sold under the name RETEN by Hercules,

quaternized or non-quaternized vinylpyrrolidone/dialkylaminoalkyl acrylate and methacrylate copolymers, such as the products sold under the name “Gafquata® by ISP, for example, “Gafquat® 734” and “Gafquat® 755”, or the products known as “Copolymer® 845, 958, and 937”. These polymers are described, for example, in French Patent Nos. 2 077 143 and 2 393 573,

fatty-chain polymers comprising a vinylpyrrolidone unit, such as the products sold under the names STYLEZE W20 and STYLEZE W10 by ISP,

dimethylaminoethyl methacrylate/vinylcaprolactam/vinylpyrrolidone terpolymers, such as the product sold under the name GAFFIX VC 713 by ISP, and

quaternized vinylpyrrolidone/dimethylaminopropylmethacrylamide copolymers, such as the products sold under the name “Gafquat® HS 100” by ISP;

(2) cationic polysaccharides, for example, those comprising quaternary ammonium, such as those described in U.S. Pat. Nos. 3,589,578 and 4,031,307, such as guar gums comprising trialkylammonium cationic groups. Such products are sold, for example, under the trade names JAGUAR C13 S, JAGUAR C 15, and JAGUAR C 17 by Meyhall;

(3) quaternary copolymers of vinylpyrrolidone and of vinylimidazole;

(4) chitosans and salts thereof, for example, chitosan acetate, lactate, glutamate, gluconate, and pyrrolidonecarboxylate. Examples of such compounds include, but are not limited to, chitosan having a degree of deacetylation of 90.5% by weight, sold under the name KYTAN BRUT STANDARD by Aber Technologies, and chitosan pyrrolidonecarboxylate sold under the name Kytamer® PC by Amerchol;

(5) cationic cellulose derivatives such as copolymers of cellulose or of cellulose derivatives grafted with a water-soluble monomer comprising a quaternary ammonium, for example, the compounds described in U.S. Pat. No. 4,131,576, such as hydroxyalkylcelluloses, for instance hydroxymethyl-, hydroxyethyl- and hydroxypropyl-celluloses grafted, e.g., with methacryloyloxyethyltrimethylammonium, methacrylamidopropyltrimethylammonium, and/or dimethyidiallylammonium salts. Examples of commercial products corresponding to this definition include, for example, the products sold under the names “Celquat L 200” and “Celquat H 100” by National Starch.

Suitable amphoteric fixative polymers that may be used herein may be chosen from polymers comprising units B and C distributed randomly in the polymer chain, wherein B is chosen from units derived from a monomer comprising at least one basic nitrogen atom and C is chosen from units derived from an acid monomer comprising at least one group chosen from carboxylic and sulphonic groups, or alternatively, B and C may be chosen from groups derived from carboxybetaine and sulphobetaine zwitterionic monomers;

B and C may also be chosen from cationic polymer chains comprising at least one group chosen from primary, secondary, tertiary, and quaternary amine groups, in which at least one of the amine groups bears a group chosen from carboxylic and sulphonic groups connected via a hydrocarbon group, or alternatively, B and C may form part of a chain of a polymer comprising at least one α,β-dicarboxylic ethylene unit, in which one of the carboxylic groups has been reacted with a polyamine comprising at least one group chosen from primary and secondary amine groups.

In at least one embodiment of the present disclosure, the amphoteric fixative polymers may be chosen from copolymers containing acidic vinyl units and basic vinyl units, crosslinked and acylated polyaminoamides, polymers containing zwitterionic units, polymers derived from chitosan, modified (C₁-C₅)alkyl vinyl ether/maleic anhydride copolymers, amphoteric polyurethanes, and grafted amphoteric silicone polymers.

In one embodiment, the amphoteric fixative polymers may be chosen, for example, from:

(1) copolymers comprising acidic vinyl and basic vinyl units, such as those resulting from the copolymerization of a monomer derived from a vinyl compound comprising at least one carboxylic group, such as acrylic acid, methacrylic acid, maleic acid, and α-chloroacrylic acid, and a basic monomer derived from a substituted vinyl compound comprising at least one basic atom, such as dialkylaminoalkyl methacrylate and acrylate, dialkylaminoalkylmethacrylamides, and dialkylaminoalkylacrylamides. Such compounds are described, for example, in U.S. Pat. No. 3,836,537;

(2) polymers comprising units derived from:

a) at least one monomer chosen from acrylamides and methacrylamides substituted on the nitrogen atom by an alkyl group,

b) at least one acidic comonomer comprising at least one reactive carboxylic group, and

c) at least one basic comonomer, such as esters containing primary, secondary, tertiary, and quaternary amine substituents of acrylic and methacrylic acids, and the product of quaternization of dimethylaminoethyl methacrylate with dimethyl or diethyl sulphate.

Examples of N-substituted acrylamides and-methacrylamides include, but are not limited to, compounds in which the alkyl groups comprise from 2 to 12 carbon atoms, for example, N-ethylacrylamide, N-tert-butylacrylamide, N-tert-octylacrylamide, N-octylacrylamide, N-decylacrylamide, N-dodecylacrylamide, and the corresponding methacrylamides.

The acidic comonomers may be chosen, for example, from acrylic acid; methacrylic acid; crotonic acid; itaconic acid; maleic acid; fumaric acid; and/or alkyl monoesters, comprising from 1 to 4 carbon atoms, of maleic acid, maleic anhydride, fumaric acid, or fumaric anhydride.

The basic comonomers may include, but are not limited to, aminoethyl, butylaminoethyl, N,N′-dimethylaminoethyl, and N-tert-butylaminoethyl methacrylates.

Further examples of suitable copolymers include those whose CTFA (4th edition, 1991) name is Octylacrylamide/acrylates/butylaminoethyl methacrylate copolymer, such as the products sold under the name Amphomer® and Lovocryl® 47 by National Starch;

(3) crosslinked and acylated polyaminoamides partially or totally derived from polyaminoamides of formula (III): [symbol]
CO—R₁₀—CO-Z [symbol] (III)
in which

R₁₀ is chosen from divalent groups derived from a saturated dicarboxylic acid, mono- or dicarboxylic aliphatic acids comprising an ethylenic double bond, esters of a lower alkanol, comprising from 1 to 6 carbon atoms, of these acids, and groups derived from the addition reaction of any one of said acids with a bis(primary) or bis(secondary) amine, and

Z is chosen from groups derived from an entity chosen from bis(primary), mono(secondary), and bis(secondary) polyalkylene-polyamines, for example:
a) a group of formula (IV) in an amount ranging from 60 to 100 mol %:
   —NH [symbol] (CH₂)ₓ—NH [symbol] ₚ (IV)
wherein x=2 and p=2 or 3, or alternatively x=3 and p=2,
this group being derived from an entity chosen from diethylenetriamine, triethylenetetraamine, and dipropylenetriamine;
b) the group (IV) above, in an amount ranging from 0 to 40 mol %, in which x=2 and p=1 and which is derived from ethylenediamine, or the group deriving from piperazine: 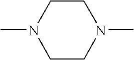
c) the group —NH(CH₂)₆—NH— deriving from hexamethylenediamine, in an amount ranging from 0 to 20 mol %,
these polyaminoamides being crosslinked by the addition reaction of a difunctional crosslinking agent chosen from epihalohydrins, diepoxides, dianhydrides, and bis-unsaturated derivatives, using the agent in an amount ranging from 0.025 to 0.35 mol of crosslinking agent per amine group of the polyaminoamide, and being acylated by the action of acrylic acid, chloroacetic acid, and/or alkane sultones, or salts thereof.

The saturated carboxylic acids may be chosen from, for example, acids comprising from 6 to 10 carbon atoms, such as adipic acid, 2,2,4-trimethyladipic acid, and 2,4,4-trimethyladipic acid; terephthalic acid; acids comprising an ethylenic double bond, for example, acrylic acid, methacrylic acid, and itaconic acid.

The alkane sultones used in the acylation may be chosen from propane sultone and butane sultone; the salts of the acylating agents may be chosen from sodium and potassium salts;

(4) polymers comprising zwifterionic units of formula (V): 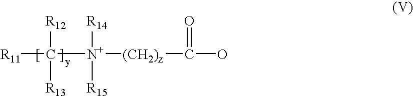
in which

R₁₁ is chosen from polymerizable unsaturated groups such as acrylate, methacrylate, acrylamide, and methacrylamide groups,

y and z, which may be identical or different, are each an integer ranging from 1 to 3,

R₁₂ and R₁₃, which may be identical or different, are chosen from hydrogen, and methyl, ethyl, and propyl groups,

R₁₄ and R₁₅, which may be identical or different, are chosen from hydrogen and alkyl groups such that the sum of the carbon atoms in R₁₄ and R₁₅ does not exceed 10.

Polymers comprising such zwitterionic units may also comprise units derived from non-zwitterionic monomers such as dimethylaminoethyl acrylate, dimethylaminoethyl methacrylate, diethylaminoethyl acrylate, diethylaminoethyl methacrylate, alkyl acrylates, alkyl methacrylates, acrylamides, methacrylamides, and vinyl acetate. A non-limiting example of a suitable copolymer is the copolymer of methyl methacrylate/methyl dimethylcarboxymethylammonioethyl methacrylate, such as the product sold under the name DIAFORMER Z301 by Sandoz;

(5) polymers derived from chitosan comprising at least one monomer unit chosen from units of formulae (D)-(F): 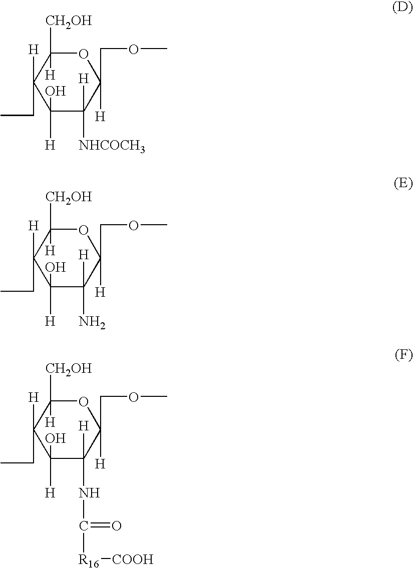
the unit (D) being present in an amount ranging from 0 to 30%, the unit (E) being present in an amount ranging from 5% to 50%, and the unit (F) being present in an amount ranging from 30% to 90%, wherein, in the unit (F), R₁₆ is chosen from groups of the following formula: 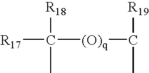
in which:

if q=0, R₁₇, R₁₈, and R₁₉, which may be identical or different, are chosen from hydrogen; methyl residues; hydroxyl residues; acetoxy residues; amino residues; monoalkylamine residues; dialkylamine residue that are optionally interrupted by at least one nitrogen atom and/or optionally substituted by at least one group chosen from amine, hydroxyl, carboxyl, alkylthio, and sulphonic groups; and alkylthio residues in which the alkyl group bears an amino residue, wherein at least one of the groups R₁₇, R₁₈, and R₁₉ is hydrogen; or

if q=1, R₁₇, R₁₈, and R₁₉ each are hydrogen, as well as the acid and base addition salts of these compounds.

(6) polymers corresponding to the general formula (VI), which are described, for example, in French Patent No. 1 400 366: 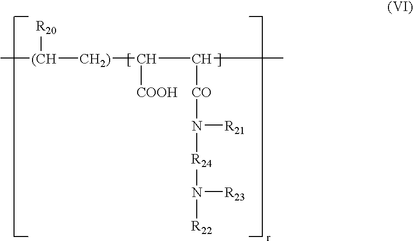
in which:

r is an integer greater than or equal to 1,

R₂₀ is chosen from hydrogen, CH₃O, CH₃CH₂O, and phenyl,

R₂₁ is chosen from hydrogen and lower alkyl groups such as methyl and ethyl,

R₂₂ is chosen from hydrogen and C₁-C₆ lower alkyl groups such as methyl and ethyl,

R₂₃ is chosen from C₁-C₆ lower alkyl groups, such as methyl and ethyl, and groups corresponding to the formula: —R₂₄—N(R₂₂)₂, wherein R₂₄ is chosen from —CH₂—CH₂—, —CH₂—CH₂—CH₂— or —CH₂—CH(CH₃)— and R₂₂ has the meanings above;

(7) polymers derived from the N-carboxyalkylation of chitosan, such as N-carboxymethylchitosan and N-carboxybutylchitosan, sold under the name “Evalsan” by Jan Dekker;

(8) amphoteric polymers of the type -D-X-D-X selected from:
a) polymers obtained by the action of chloroacetic acid or sodium chloroacetate on compounds comprising at least one unit of formula (VII):
   -D-X-D-X-D- (VII)
   wherein D is a group of the following formula: 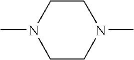
   and X is chosen from E and E′, wherein E or E′, which may be identical or different, are chosen from divalent groups that are alkylene groups with a straight or branched chain comprising up to 7 carbon atoms in the main chain, which is unsubstituted or substituted by hydroxyl groups and which may comprise, in addition to the oxygen, nitrogen, and sulphur atoms, 1 to 3 aromatic and/or heterocyclic rings; the oxygen, nitrogen, and sulphur atoms being present in a form chosen from ether, thioether, sulphoxide, sulphone, sulphonium, alkylamine, and alkenylamine groups, hydroxyl, benzylamine, amine oxide, quaternary ammonium, amide, imide, alcohol, ester, and/or urethane groups;
b) polymers of formula (VII′):
   -D-X-D-X- (VII′)

wherein D is a group of the following formula: 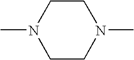
and X is chosen from E and E″, and at least once E′; wherein E has the meaning given above and E″ is a divalent group that is an alkylene group with a straight or branched chain comprising up to 7 carbon atoms in the main chain, which is unsubstituted or substituted by at least one hydroxyl group and contains at least one nitrogen atom, the nitrogen atom being substituted by an alkyl chain that is optionally interrupted by an oxygen atom and comprises at least one carboxyl function or at least one hydroxyl function and betainized by reaction with chloroacetic acid or sodium chloroacetate;

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers may also comprise other vinyl comonomers such as vinylcaprolactam.

In at least one embodiment, the amphoteric fixative polymers may be chosen from those described in class (3), for example, the copolymers whose CTFA name is Octylacrylamide/acrylates/butylaminoethyl methacrylate copolymer, such as the products sold under the names Amphomer®, Amphomero® LV 71 and Lovocryl® 47 by National Starch and those described in class (4), for example, the copolymers of methyl methacrylate/methyl dimethylcarboxymethylammonioethyl methacrylate, sold, for example, under the name DIAFORMER Z301 by Sandoz.

The nonionic fixative polymers that may be used according to the present disclosure may be chosen, for example, from:

polyalkyloxazolines;

vinyl acetate homopolymers;

vinyl acetate copolymers, for instance copolymers of vinyl acetate and of acrylic ester; copolymers of vinyl acetate and of ethylene, or copolymers of vinyl acetate and of maleic ester, for example of dibutyl maleate;

acrylic ester homopolymers and copolymers, for instance copolymers of alkyl acrylates and of alkyl methacrylates, such as the products sold by Rohm & Haas under the names Primal® AC-261 K and Eudragit® NE 30 D, by BASF under the name 8845, or by Hoechst under the name Appretan® N9212;

acrylonitrile copolymers and copolymers of a nonionic monomer chosen, for example, from butadiene and alkyl (meth)acrylates, for example, the products sold under the name CJ 0601 B by Rohm & Haas;

styrene homopolymers;

styrene copolymers, for instance copolymers of styrene and of an alkyl (meth)acrylate, such as the products Mowilith® LDM 6911, Mowilith® DM 611, and Mowilith® LDM 6070 sold by Hoechst, and the products Rhodopas® SD 215 and Rhodopas® DS 910 sold by Rhône-Poulenc; copolymers of styrene, of alkyl methacrylate and of alkyl acrylate; copolymers of styrene and of butadiene; or copolymers of styrene, of butadiene and of vinylpyridine;

polyamides;

vinyllactam homopolymers other than vinylpyrrolidone homopolymers, such as the polyvinylcaprolactam sold under the name Luviskol® Plus by BASF; and

vinyllactam copolymers such as a poly(vinylpyrrolidone/vinyllactam) copolymer sold under the trade name Luvitec® VPC 55K65W by BASF, poly(vinylpyrrolidone/vinyl acetate) copolymers, such as those sold under the name PVPVA® S630L by ISP, Luviskol® VA 73, VA 64, VA 55, VA 37, and VA 28 by BASF; and poly(vinylpyrrolidone/vinyl acetate/vinyl propionate) terpolymers, for instance the product sold under the name Luviskol® VAP 343 by BASF.

In at least one aspect of the present disclosure, the alkyl groups of the nonionic polymers mentioned above may comprise from 1 to 6 carbon atoms.

According to one embodiment of the present disclosure, the fixative polymers may be of the grafted silicone type, comprising a polysiloxane portion and a portion comprising a non-silicone organic chain, one of the two portions constituting the main chain of the polymer and the other being grafted onto said main chain.

These polymers are described, for example, in European Patent Nos. 0 412 704, 0 412 707, 0 640 105, 0 582 152, International Publication Nos. WO 95/00578 and WO 93/23009, and U.S. Pat. Nos. 4,693,935, 4,728,571, and 4,972,037. These polymers may be chosen from amphoteric, anionic, and nonionic polymers. In one embodiment, the polymers are chosen from anionic and nonionic polymers.

Such polymers include, for example, copolymers that can be obtained by free radical polymerization from the monomer mixture formed from:
a) 50% to 90% by weight of tert-butyl acrylate;
b) 0 to 40% by weight of acrylic acid;
c) 5% to 40% by weight of a silicone macromer of formula: 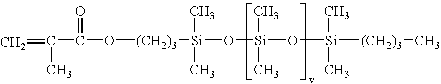
   in which v is a number ranging from 5 to 700, the weight percentages being calculated relative to the total weight of the monomers.

Other examples of grafted silicone polymers include, but are not limited to, polydimethylsiloxanes (PDMSs) onto which are grafted, via a thiopropylene-type connecting chain, mixed polymer units of the poly(meth)acrylic acid type and of the polyalkyl (meth)acrylate type and polydimethylsiloxanes (PDMSs) onto which are grafted, via a thiopropylene-type connecting chain, polymer units of the polyisobutyl (meth)acrylate type.

Yet another non-limiting example of a suitable silicone fixative polymer is the product Luviflex® Silk, sold by BASF.

In at least one embodiment, functionalized or non-functionalized, silicone or non-silicone, cationic, nonionic, anionic, or amphoteric polyurethanes and mixtures thereof with a molecular weight of less than 400 000 may also be used as fixative polymers.

Additional examples of suitable fixative polyurethanes include, but are not limited to, the products sold under the names Luviset Pur® and Luviset® Si Pur by BASF.

The at least one additional fixative polymer may be present in the composition in an amount ranging from 0.1% to 20%, for example, from 0.5% to 10% by weight relative to the total weight of the composition.

Thickening Polymer

The compositions according to the present patent application may also contain as an additional cosmetic adjuvant at least one additional thickening polymer, also known as a “rheology modifier”, other than the polyurethanes disclosed herein.

The at least one rheology modifier may be chosen from fatty acid amides (for example, coconut monoethanolamide, coconut diethanolamide, and oxyethylenated carboxylic acid alkyl ether monoethanolamide), cellulose-based thickeners (for example, hydroxyethylcellulose, hydroxypropylcellulose, and carboxymethylcellulose), guar gums and derivatives thereof, such as hydroxypropyl guar gum, gums of microbial origin (for example, xanthan gum and scleroglucan gum), crosslinked acrylic acid or acrylamidopropanesulphonic acid homopolymers, and associative polymers.

The at least one associative polymer that may be used in accordance with the present disclosure may be water-soluble polymers that are capable, in an aqueous medium, of reversibly associating with each other or with other molecules.

The chemical structure of the associative polymer may comprise hydrophilic zones and hydrophobic zones characterized by at least one fatty chain. In at least one embodiment, the associative polymers may be chosen from anionic, cationic, amphoteric, and nonionic polymers.

The at least one additional thickening polymer may be present in the composition in an amount ranging from 0.01% to 20%, for example, from 0.05% to 10% by weight relative to the total weight of the composition.

Silicones, Silicone Fatty Substances, and Non-Silicone Fatty Substances

The compositions according to the present disclosure may also contain as an additional cosmetic adjuvant at least one compound selected from silicones, silicone fatty substances, and non-silicone fatty substances.

The at least one silicone may be chosen from linear, cyclic, branched, unbranched, volatile, and non-volatile silicones. They may be in a form chosen from soluble, dispersed, and microdispersed form and may be, for example, in the form of oils, resins, and gums. In at least one embodiment of the present disclosure, the silicones may be polyorganosiloxanes that are insoluble in the cosmetically acceptable medium.

Suitable polyorganosiloxanes are defined, for example, in Walter Noll's “Chemistry and Technology of Silicones,” Academic Press (1968). The polyorganosiloxanes may be volatile or non-volatile.

Non-limiting examples of volatile silicones include those having a boiling point ranging from 60° C. to 260° C., for example:

(i) cyclic silicones comprising from 3 to 7, for example, from 4 to 5 silicon atoms. These include, for example, octamethylcyclotetrasiloxane sold in particular under the name VOLATILE SILICONE 7207 by Union Carbide and SILBIONE 70045 V 2 by Rhodia, decamethylcyclopentasiloxane sold under the name VOLATILE SILICONE 7158 by Union Carbide, SILBIONE 70045 V 5 by Rhodia, and mixtures thereof.

Such cyclic silicones also include cyclocopolymers of the dimethylsiloxane/methylalkylsiloxane type, such as VOLATILE SILICONE FZ 3109 sold by Union Carbide, having the chemical structure:

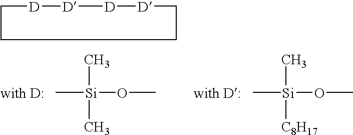

Mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane, may also be used;

(ii) linear volatile silicones comprising from 2 to 9 silicon atoms and having a viscosity of less than or equal to 5×10⁻⁶ m²/s at 25° C., for example, decamethyltetrasiloxane, sold for example under the name SH 200 by Toray Silicone. Silicones belonging to this category are also described, for example, in Todd & Byers “Volatile Silicone Fluids for Cosmetics,” Cosmetics and Toiletries, 91: 27-32 (January 1976).

Non-limiting examples of non-volatile silicones include polyalkylsiloxanes, polyarylsiloxanes, polyalkylarylsiloxanes, silicone gums and resins, polyorganosiloxanes modified with organofunctional groups, and mixtures thereof.

Organomodified silicones that may be used in accordance with the present disclosure include silicones as defined above that also contain in their structure at least one organofunctional group attached via a hydrocarbon-based group.

Examples of organomodified silicones include, but are not limited to, polyorganosiloxanes comprising:

polyethyleneoxy and/or polypropyleneoxy groups optionally comprising at least one C₆-C₂₄ alkyl group, such as the products known as dimethicone copolyol and sold, for example, by Dow Corning under the name DC 1248 and by Union Carbide as the oils Silwet® L 722, L 7500, L 77, and L 711 and the product known as (C₁₂)alkylmethicone copolyol and sold by Dow Corning under the name Q2 5200;

substituted and unsubstituted amine groups, such as the products sold under the name GP 4 Silicone Fluid and GP 7100 by Genesee, or the products sold under the names 02 8220 and Dow Corning 929 and 939 by Dow Corning. In one embodiment, the substituted amine groups may be chosen from C₁-C₄ aminoalkyl groups;

thiol groups, such as the products sold under the names “GP 72 A” and “GP 71” from Genesee;

alkoxylated groups, such as the product sold under the name “Silicone Copolymer F-755” by SWS Silicones and Abil Wax® 2428, 2434, and 2440 by Goldschmidt;

hydroxyl groups, such as the polyorganosiloxanes containing at least one hydroxyalkyl function, described, for example, in French Patent Application No. 85/16334;

acyloxyalkyl groups, for example, the polyorganosiloxanes described in U.S. Pat. No. 4,957,732;

anionic groups of the carboxylic acid type, for example, those present in the products described in European Patent No. 0 186 507 from Chisso Corporation, and of the alkylcarboxylic type, such as those present in the product X-22-3701 E from Shin-Etsu; 2-hydroxyalkyl sulphonate; and 2-hydroxyalkyl thiosulphate, such as the products sold by Goldschmidt under the names “Abil® S201” and “Abil® S255”; and

hydroxyacylamino groups, such as the polyorganosiloxanes described in European Patent No. 0 342 834, for example, the product Q2-8413 from Dow Corning.

Silicone oils that may be used in the compositions according to the present disclosure may be volatile and non-volatile polymethylsiloxanes containing a linear or cyclic silicone chain, which are liquid or pasty at room temperature, for example, cyclopolydimethylsioxanes (cyclomethicones) such as cyclohexasiloxane; polydimethylsiloxanes comprising at least one group chosen from alkyl, alkoxy, and phenyl groups, which are pendent or at the end of a silicone chain, these groups comprising from 2 to 24 carbon atoms; phenylsilicones, for instance phenyltrimethicones, phenyldimethicones, phenyltrimethylsiloxydiphenylsiloxanes, diphenyldimethicones, diphenylmethyldiphenyltrisiloxanes, 2-phenylethyltrimethyl siloxysilicates, and polymethylphenylsiloxanes; and mixtures thereof.

The silicone gums that may be used in the compositions according to the present disclosure include polydiorganosiloxanes with a high molecular mass, for example, ranging from 200 000 to 5 000 000 g/mol, used alone or as a mixture in a solvent chosen from volatile silicones, polydimethylsiloxane oils, polymethylphenylsiloxane oils, polydiphenyldimethylsiloxane oils, isoparaffins, methylene chloride, pentane, hydrocarbons, and mixtures thereof.

In at least one embodiment of the present disclosure, the silicone gum has a molecular weight of less than 1 500 000. Examples of silicone gums include, but are not limited to, polydimethylsiloxanes, polyphenylmethylsiloxanes, poly(diphenylsiloxanedimethylsiloxanes), poly(dimethylsiloxanemethylvinylsiloxanes), poly(dimethylsiloxanephenylmethylsiloxanes), and poly(diphenylsiloxanedimethylsiloxanemethylvinylsiloxanes).

According to one embodiment, these silicone gums may be terminated at a chain end with a group chosen from trimethylsilyl and dimethylhydroxysilyl groups.

The silicone resins that may be used in the compositions according to the present disclosure may be crosslinked siloxane systems comprising units chosen from R₂SiO_{2/2}, RSiO_{3/2}, and SiO_{4/2}, in which R is chosen from hydrocarbon-based group comprising from 1 to 6 carbon atoms or a phenyl group. In one embodiment, R may be chosen from lower (C₁-C₆) alkyl and phenyl radicals.

The at least one non-silicone fatty substance that may be used in the compositions according to the present disclosure may be chosen from all natural and synthetic, organic and mineral, non-silicone oils, waxes, and resins.

As used herein, an oil is a lipophilic compound that is liquid at room temperature (about 25° C.), with a reversible solid/liquid state change. Animal oils and plant oils generally comprise propane-1,2,3-triol triesters as essential constituents.

Oils that may be used in the composition of the present disclosure include, but are not limited to:

hydrocarbon oils of animal origin, such as perhydrosqualene;

hydrocarbon oils of plant origin, such as liquid triglycerides of fatty acids comprising from 4 to 10 carbon atoms, for instance heptanoic acid triglycerides and octanoic acid triglycerides, sunflower oil, maize oil, soybean oil, marrow oil, grapeseed oil, sesame oil, hazelnut oil, apricot oil, macadamia oil, arara oil, castor oil, avocado oil, caprylic/capric acid triglycerides, for instance those sold by Stearineries Dubois or those sold under the names MIGLYOL 810, 812, and 818 by Dynamit Nobel, jojoba oil, and shea butter oil;

synthetic esters and synthetic ethers, for example, of fatty acids, for instance oils of formulae R⁶COOR⁷ and R⁶OR⁷, in which R⁶ is chosen from fatty acid residues comprising from 8 to 29 carbon atoms and R⁷ is chosen from branched or unbranched hydrocarbon chains comprising from 3 to 30 carbon atoms, for example, purcellin oil, isononyl isononanoate, isopropyl myristate, 2-ethylhexyl palmitate, 2-octyldodecyl stearate, 2-octyldodecyl erucate, and isostearyl isostearate; hydroxylated esters such as isostearyl lactate, octyl hydroxystearate, octyldodecyl hydroxystearate, diisostearyl malate, triisocetyl citrate, fatty alcohol heptanoates, fatty alcohol octanoates, and fatty alcohol decanoates; polyol esters, for instance propylene glycol dioctanoate, neopentyl glycol diheptanoate, and diethylene glycol diisononanoate; and pentaerythritol esters, for instance pentaerythrityl tetraisostearate;

linear or branched hydrocarbons of mineral or synthetic origin, such as volatile or non-volatile liquid paraffins, and derivatives thereof, petroleum jelly, polydecenes, and hydrogenated polyisobutene such as parleam oil;

fluid fatty alcohols comprising from 8 to 26 carbon atoms, for instance octyldodecanol, 2-butyloctanol, oleyl alcohol, linoleyl alcohol, and linolenyl alcohol;

alkoxylated, for example, ethoxylated, fatty alcohols such as oleth-12;

partially hydrocarbon-based fluoro oils, for instance those described in Japanese Publication No. 2 295 912. Further examples of fluoro oils include, but are not limited to, perfluoromethylcyclopentane and perfluoro-1,3-dimethylcyclohexane, sold under the names “Flutec PC1®” and “Flutec PC3®” by BNFL Fluorochemicals; perfluoro-1,2-dimethylcyclobutane; perfluoroalkanes such as dodecafluoropentane and tetradecafluorohexane, sold under the names “PF 5050®” and “PF 5060®” by 3M, and bromoperfluorooctyl sold under the name “Foralkyl®” by Atochem; nonafluoromethoxybutane sold under the name “MSX 4518®” by 3M and nonafluoroethoxyisobutane; and perfluoromorpholine derivatives, such as the 4-trifluoromethylperfluoromorpholine sold under the name “PF 5052®” by 3M.

As used herein, the term “hydrocarbon oil” means any oil predominantly comprising carbon and hydrogen atoms, and possibly ester, ether, fluoro, carboxylic acid, and/or alcohol groups.

As used herein, a wax is a lipophilic compound that is solid at room temperature (about 25° C.), with a reversible solid/liquid state change, having a melting point of greater than about 4⁰0C and possibly up to 200° C., and having an anisotropic crystal organization in the solid state. As essential constituents, animal and plant waxes generally comprise carboxylic acid esters of long-chain alcohols. In general, the size of the wax crystals is such that crystals scatter and/or diffuse light, giving the composition comprising them a more or less opaque, cloudy appearance. By raising the wax to its melting point, it is possible to make the wax miscible with oils to form a microscopically homogeneous mixture, but on returning the temperature of the mixture to room temperature, a microscopically and macroscopically detectable recrystallization of the wax in the oils of the mixture may be obtained (opalescence).

Waxes that may be used in accordance with the present disclosure include, but are not limited to, waxes of animal origin such as beeswax, spermaceti, lanolin wax, and lanolin derivatives; plant waxes such as sunflower wax, rice wax, potato wax, carnauba wax, candelilla wax, ouricury wax, Japan wax, cocoa butter, cork fiber wax, and sugarcane wax; mineral waxes, for example paraffin wax, petroleum jelly wax, lignite wax, microcrystalline waxes, ceresin, and ozokerite; synthetic waxes such as polyethylene waxes and Fischer-Tropsch waxes; and mixtures thereof.

The at least one compound chosen from silicones, silicone fatty substances, and non-silicone fatty substances may be present in the composition in an amount ranging from 0.01% to 20%, for example, from 0.05% to 10% by weight relative to the total weight of the composition.

The styling composition according to the present disclosure may also comprise at least one additive chosen from non-ionic surfactants, anionic surfactants, cationic surfactants, amphoteric surfactants; additional nonionic, anionic, cationic, and amphoteric polymers other than the fixative polymers used in the compositions according to the present disclosure; ceramides and pseudoceramides; vitamins and provitamins, including panthenol; silicone and non-silicone, water-soluble and lipid-soluble sunscreens; fillers; solid particles; uncoloured mineral and organic pigments; nacreous agents; opacifiers; flakes; active particles; dyes; sequestrants; plasticizers; solubilizers; acidifying agents; alkalifying agents; neutralizing agents; organic and inorganic thickeners; antioxidants; hydroxy acids; penetrants; fragrances; and preservatives.

Thickeners

Natural thickeners suitable for use in the compositions disclosed herein include, for example, xanthan gum, scleroglucan gum, gellan gum, rhamsan gum, alginates, maltodextrin, starch and its derivatives, karaya gum, carob flour, and guar gums.

Suitable synthetic thickeners include, for example, polymers and copolymers of acrylic and/or methacrylic acid, such as acrylic acid/ethyl acrylate copolymers and carboxyvinyl polymers. Examples of such polymers and copolymers include, but are not limited to “carbomers” (CTFA) sold by Goodrich under the name CARBOPOL, the polyglyceryl methacrylate sold by Guardian under the name LUBRAGEL, and the polyglyceryl acrylate sold under the name HISPAGEL by Hispano Chimica.

Other acrylic compounds include, for example, copolymers of acrylic or methacrylic acid comprising at least one C₁-C₃₀ alkyl acrylate unit and/or at least one urethane unit optionally substituted by a fatty chain, such as PEMULEN TR1 (Goodrich), VISCOPHOBE DB 1000 (Union Carbide), and ACRYSOL 44 or 46 (Rohm & Haas).

In one embodiment, polyethylene glycols (PEG) and derivatives thereof may also be used as thickeners in the compositions according to the present disclosure.

In another embodiment, thickening polyacrylamides may be used as thickeners. These may be chosen, for example, from:

crosslinked 2-acrylamido-2-methylpropanesulphonic homopolymers,

optionally crosslinked acrylamide-ammonium acrylate copolymers,

optionally crosslinked acrylamide (or methacrylamide)-methacryloyloxyethyl-trimethylammonium chloride copolymers, and

optionally crosslinked, fully or partly neutralized acrylamide-2-acrylamido-2-methylpropanesulphonic acid copolymers.

Suitable crosslinked acrylamide/ammonium acrylate copolymers include, but are not limited to, acrylamide/ammonium acrylate (5/95 by weight) copolymers crosslinked with a crosslinking agent comprising olefinic polyunsaturation, such as divinylbenzene, tetraallyloxyethane; methylenebisacrylamide; diallyl ether; polyallyl polyglyceryl ethers; and allyl ethers of alcohols of the sugar series, such as erythritol, pentaerythritol, arabitol, mannitol, sorbitol, and glucose.

Similar copolymers and their preparation are described, for example, in French Patent No.2 416 723 and in U.S. Pat. Nos. 2,798,053 and 2,923,692.

Such crosslinked copolymers may be used in the form of a water-in-oil emulsion comprising approximately 30% by weight of said copolymer, 25% by weight of liquid paraffin, 4% by weight of a mixture of sorbitan stearate and a hydrophilic ethoxylated derivative, and 41% by weight of water. An emulsion of this kind is sold, for example, under the name “Bozepol C” by Hoechst.

The acrylamide-2-acrylamido-2-methylpropanesulphonic acid copolymers used in accordance with the present disclosure may include copolymers which are crosslinked by a compound containing olefinic polyunsaturation, such as those mentioned above, and are fully or partially neutralized by a neutralizing agent, for example, sodium hydroxide, potassium hydroxide, aqueous ammonia, or an amine such as triethanolamine or monoethanolamine.

These copolymers may be prepared by copolymerizing acrylamide and sodium 2-acrylamido-2-methylpropanesulphonate by a free-radical route using initiators of azobisisobutyronitrile type and by precipitation from an alcohol such as tert-butanol.

In one embodiment of the present disclosure, the copolymers may be obtained by copolymerizing 55 to 70 mol % of acrylamide and 30 to 45 mol % of sodium 2-acrylamido-2-methylpropanesulphonate. The crosslinking agent may be used at concentrations ranging from 10⁻⁴ to 4×10⁻⁴ mol per mole of the monomer mixture.

These copolymers may be incorporated into the compositions of the present disclosure in the form of water-in-oil emulsions containing 35% to 40% by weight of this copolymer, 15% to 25% by weight of a mixture of C₁₂-C₁₃ isoparaffinic hydrocarbons, 3% to 8% by weight of polyethylene glycol lauryl ether containing 7 mol of ethylene oxide, and water. An emulsion of this kind is sold, for example, under the name “Sepigel 305” by SEPPIC.

In at least one embodiment, the crosslinked acrylamide-methacryloyloxy-ethyltrimethylammonium chloride copolymer used in accordance with the present disclosure may be chosen from copolymers obtained by copolymerizing acrylamide and dimethylaminoethyl methacrylate quaternized with methyl chloride, followed by crosslinking with an olefinically unsaturated compound, for example, methylenebisacrylamide.

A crosslinked acrylamide/methacryloyloxyethyltrimethylammonium chloride (approximately 50/50 by weight) copolymer in the form of a dispersion comprising 50% by weight of said copolymer in mineral oil may also be used in the compositions described herein. This dispersion is sold, for example, under the name “Salcare SC92” by Allied Colloids.

Non-crosslinked methacrylamide-methacryloyloxyethyltrimethylammonium chloride copolymers include, for example, the products sold under the trade names Rohagit KF400 and KF720 by Rohm & Haas.

Surfactants

Suitable surfactants in accordance with the present disclosure include, but are not limited to:

(i) Anionic Surfactants:

In the context of the present disclosure, the nature of the anionic surfactant does not represent a truly critical factor.

Thus, examples of anionic surfactants which may be used in accordance with the present disclosure, alone or as mixtures, include, but are not limited to, salts (for example, alkali metal salts, such as sodium salts; ammonium salts; amine salts; amino alcohol salts; and alkaline earth metal salts, such as magnesium salts) of compounds chosen from alkyl sulphates, alkyl ether sulphates, alkylamido ether sulphates, alkylaryl polyether sulphates, monoglyceride sulphates; alkylsulphonates, alkyl phosphates, alkylamidesulphonates, alkylarylsulphonates, α-olefinsulphonates, paraffinsulphonates; alkylsulphosuccinates, alkyl ether sulphosuccinates, alkylamide-sulphosuccinates; alkylsulphosuccinamates; alkyl sulphoacetates; alkyl ether phosphates; acylsarcosinates; acylisethionates, and N-acyltaurates, wherein the alkyl and acyl radicals of all of these various compounds comprises, for example, from 12 to 20 carbon atoms, and the aryl radicals may be chosen from phenyl and benzyl groups. Further examples of anionic surfactants include, but are not limited to, fatty acid salts such as the salts of oleic, ricinoleic, palmitic, and stearic acids; coconut oil acid and hydrogenated coconut oil acid; and acyl lactylates in which the acyl radical comprises from 8 to 20 carbon atoms. Weakly anionic surfactants may also be used, such as alkyl-D-galactoside uronic acids and their salts, polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, for example, those comprising from 2 to 50 ethylene oxide groups, and mixtures thereof.

In at least one embodiment of the present disclosure, the anionic surfactant may be chosen from alkyl sulphate salts, alkyl ether sulphate salts, and mixtures thereof.

(ii) Nonionic Surfactants:

Suitable nonionic surfactants include, for example, compounds that are well known in the art (see, for example, M. R. Porter, “Handbook of Surfactants,” Blackie & Son (Glasgow and London) pp. 116-178 (1991)) and, in the context of the present disclosure, their nature is not a critical feature. Thus, examples of nonionic surfactants include, but are not limited to, polyethoxylated, polypropoxylated, and polyglycerolated fatty acids; alkylphenols; α-diols and alcohols having a fatty chain comprising, for example, from 8 to 18 carbon atoms, wherein the number of ethylene oxide or propylene oxide groups may range, for example, from 2 to 50, and wherein the number of glycerol groups may range, for example, from 2 to 30. Further examples of suitable nonionic surfactants include, for example, copolymers of ethylene oxide and of propylene oxide, condensates of ethylene oxide and of propylene oxide with fatty alcohols; polyethoxylated fatty amides comprising, for example, from 2 to 30 mol of ethylene oxide; polyglycerolated fatty amides comprising on average 1 to 5, for example, 1.5 to 4 glycerol groups; oxyethylenated fatty acid esters of sorbitan comprising from 2 to 30 mol of ethylene oxide; fatty acid esters of sucrose; fatty acid esters of polyethylene glycol; alkylpolyglycosides; N-alkylglucamine derivatives; and amine oxides such as (C₁₀-C₁₄)alkylamine oxides or N-acylaminopropylmorpholine oxides.

(iii) Amphoteric Surfactants:

Suitable amphoteric surfactants, whose nature is not a critical feature in the context of the present disclosure, may be chosen from, for example, aliphatic secondary and tertiary amine derivatives in which the aliphatic radical is a linear or branched chain comprising from 8 to 22 carbon atoms and comprising at least one water-soluble anionic group (for example, carboxylate, sulphonate, sulphate, phosphate, and phosphonate), such as (C₈-C₂₀)alkylbetaines, sulphobetaines, (C₈-C₂₀)alkylamido(C₁-C₆)alkylbetaines, and (C₈-C₂₀)alkylamido(C₁-C₆)alkylsulphobetaines.

Non-limiting examples of amine derivatives include, but are not limited to, the products sold under the name Miranol®, described in U.S. Pat. Nos. 2,528,378, and 2,781,354 and having the following formulae (2) and (3):
R₂—CONHCH₂CH₂—N(R₃)(R₄)(CH₂COO—) (2)
in which:

R₂ is chosen from alkyl radical derived from an acid R₂—COOH present in hydrolysed coconut oil and heptyl, nonyl, and undecyl radicals,

R₃ is chosen from β-hydroxyethyl groups; and

R₄ is chosen from carboxymethyl groups;

and
R_{2′}—CONHCH₂CH₂—N(B)(C) (3)
in which:

B is —CH₂CH₂OX′,

C is —(CH₂)_{z}—Y′, wherein z=1 or 2,

X′ is chosen from —CH₂CH₂—COOH and hydrogen,

Y′ is chosen from —COOH and —CH₂—CHOH—SO₃H, and

R₂ is chosen from alkyl radicals of an acid R₉—COOH present in coconut oil or in hydrolysed linseed oil, alkyl radicals, for example, radicals chosen from C₇, C₉, C₁₁, and C₁₃ alkyl radicals, C₁₇ alkyl radicals and their iso forms, and unsaturated C₁₇ radicals.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Capryloamphodipropionate, Lauroamphodipropionic acid, and Cocoamphodipropionic acid.

A non-limiting example of a suitable amphoteric surfactant is cocoamphodiacetate, sold under the trade name Miranol® C2M concentrate by Rhodia.

Mixtures of surfactants may also be used in the compositions in accordance with the present disclosure, for example, mixtures of anionic surfactants and of amphoteric and mixtures of anionic surfactants and of nonionic surfactants. In at least one embodiment, the compositions disclosed herein may comprise a mixture comprising at least one anionic or nonionic surfactant and at least one amphoteric surfactant.

Such mixtures may include anionic surfactants chosen from sodium, triethanolamine, and ammonium (C₁₂-C₁₄)alkyl sulphates; sodium, triethanolamine and ammonium (C₁₂-C₁₄)alkyl ether sulphates oxyethylenated with 2.2 mol of ethylene oxide; sodium cocoyl-isethionate; sodium α-(C₁₄-C₁₆)olefinsulphonate; and mixtures thereof, and

either an amphoteric surfactant such as the amine derivatives known as disodium cocoamphodipropionate and sodium cocoamphopropionate sold, for example, by Rhodia under the trade name Miranol® C2M CONC as an aqueous solution containing 38% active substance, or under the name Miranol® C32;

or an amphoteric surfactant of zwitterionic type such as alkylbetaines, for example, the cocoylbetaine sold under the name Dehyton® AB 30 as an aqueous solution containing 32% a.s. by Henkel and alkylamidobetaines such as Tegobetaine® F50 sold by Goldschmidt.

(iv) Cationic Surfactants

The composition according to the present disclosure may comprise at least one cationic surfactant, which is well known in the art and whose nature is not a critical feature, such as optionally polyoxyalkylenated primary, secondary, and tertiary fatty amine salts, quaternary ammonium salts, and mixtures thereof.

Examples of suitable quaternary ammonium salts include, but are not limited to:

salts of formula (VIII) below: 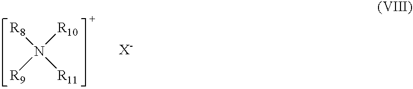
in which:

R₈ to R₁₁, which may be identical or different, are chosen from linear or branched aliphatic radicals comprising from 1 to 30 carbon atoms, and aromatic radicals such as aryl and alkylaryl radicals. The aliphatic radicals may comprise at least one heteroatom, for example, oxygen, nitrogen, sulphur, and halogens. The aliphatic radicals may be chosen, for example, from alkyl, alkoxy, polyoxy(C₂-C₆)alkylene, alkylamide, (C₁₂-C₂₂)alkylamido(C₂-C₆)alkyl, (C₁₂-C₂₂)alkyl acetate, and hydroxyalkyl radicals, comprising from 1 to 30 carbon atoms; and

X is an anion chosen from halides, phosphates, acetates, lactates, (C₂-C₆)alkyl sulphates, alkylsulphonates, and alkylarylsulphonates;

quaternary ammonium salts of imidazoline, for example, those of formula (IX) below: 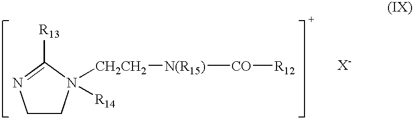
in which:

R₁₂ is chosen from alkenyl and alkyl radicals comprising from 8 to 30 carbon atoms, for example, tallow fatty acid derivatives,

R₁₃ is chosen from hydrogen, C₁-C₄ alkyl radicals, and alkenyl and alkyl radicals comprising from 8 to 30 carbon atoms,

R₁₄ is chosen from C₁-C₄ alkyl radicals,

R₁₅ is chosen from hydrogen and C₁-C₄ alkyl radicals, and

X⁻ is an anion chosen from halides, phosphates, acetates, lactates, alkyl sulphates, alkylsulphonates, and alkylarylsulphonates.

In at least one embodiment of the present disclosure, R₁₂ and R₁₃ may comprise a mixture of alkenyl and alkyl radicals comprising from 12 to 21 carbon atoms, for example tallow fatty acid derivatives, R₁₄ may be methyl, and R₁₅ may be hydrogen. Such a product is sold, for example, under the name Rewoquat® W 75 by Rewo;

diquaternary ammonium salts of formula (X): 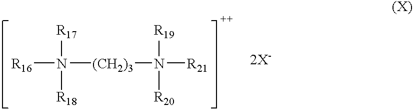
in which:

R₁₆ is chosen from aliphatic radicals comprising from 16 to 30 carbon atoms,

R₁₇, R₁₈, R₁₉, R₂₀, and R₂₁, which may be identical or different, are chosen from hydrogen and alkyl radicals comprising from 1 to 4 carbon atoms, and

X is an anion chosen from halides, acetates, phosphates, nitrates, and methyl sulphates. A non-limiting example of such diquaternary ammonium salts is propane tallow diammonium dichloride;

quaternary ammonium salts comprising at least one ester function, such as those of formula (XI) below: 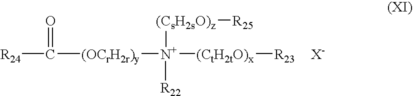
in which:

R₂₂ is chosen from C₁-C₆ alkyl radicals, C₁-C₆ hydroxyalkyl radicals, and C₁-C₆ dihydroxyalkyl radicals;

R₂₃ is chosen from 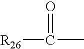
radicals, linear or branched, saturated or unsaturated C₁-C₂₂ hydrocarbon radicals R₂₇, and hydrogen,

R₂₅ is chosen from 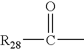
radicals, linear or branched, saturated or unsaturated C₁-C₆ hydrocarbon radicals R₂₉, and hydrogen,

R₂₄, R₂₆, and R₂₈, which are identical or different, are chosen from linear or branched, saturated or unsaturated C₇-C₂₁ hydrocarbon radicals;

r, s, and t, which are identical or different, are integers ranging from 2 to 6;

y is an integer ranging from 1 to 10;

x and z, which are identical or different, are integers ranging from 0 to 10; and

X⁻ is a simple or complex, organic or inorganic anion;

with the proviso that the sum x+y+z ranges from 1 to 15, that when x is 0, R₂₃ denotes R₂₇, and that when z is 0, R₂₅ denotes R₂₉.

In at least one embodiment, the R₂₂ alkyl radicals may be linear or branched. In another embodiment, the R₂₂ alkyl radicals may be linear. In yet another embodiment, R₂₂ may be chosen from methyl, ethyl, hydroxyethyl, and dihydroxypropyl radicals. In still a further embodiment, R₂₂ may be chosen from methyl and ethyl radicals.

According to at least one embodiment of the present disclosure, the sum x+y+z may range from 1 to 10.

When R₂₃ is a hydrocarbon radical R₂₇, it may be long and comprise from 12 to 22 carbon atoms, or it may be short and comprise from 1 to 3 carbon atoms.

When R₂₅ is a hydrocarbon radical R₂₉, it may comprise from 1 to 3 carbon atoms.

R₂₄, R₂₆ and R₂₈, which are identical or different, may be chosen from linear or branched, saturated or unsaturated C₁₁-C₂₁ hydrocarbon radicals, for example, linear or branched, saturated or unsaturated, C₁₁-C₂₁ alkyl and alkenyl radicals.

In at least one embodiment of the present disclosure, x and z, which are identical or different, may be equal to 0 or 1. In another embodiment, y may be equal to 1. In a further embodiment, r, s, and t, which are identical or different, may be equal to 2 or 3. In still a further embodiment, r, s, and t may be equal to 2.

The anion X may be chosen from halides (for example, chloride, bromide, and iodide) and alkyl sulphates, such as methyl sulphate. However, any other anion that is compatible with the ester-functional ammonium may be used, such as methanesulphonate, phosphate, nitrate, tosylate, and anions derived from organic acids, such as acetate and lactate. In at least one embodiment, the anion X⁻ may be chosen from chloride and methyl sulphate.

The ammonium salts used in the composition according to the present disclosure may include, for example, those of formula (XI) in which:

R₂₂ is chosen from methyl and ethyl radicals,

x and y are equal to 1;

z is equal to 0 or 1;

r, s, and t are equal to 2;

R₂₃ is chosen from hydrogen and 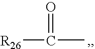
methyl, ethyl, and C₁₄-C₂₂ hydrocarbon radicals;

R₂₅ is chosen from hydrogen and 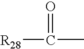
radicals;

R₂₄, R₂₆, and R₂₈, which may be identical or different, are chosen from linear or branched, saturated or unsaturated C₁₃-C₁₇ hydrocarbon radicals, for example, linear or branched, saturated or unsaturated C₁₃-C₁₇ alkyl and alkenyl radicals.

In at least one embodiment, the hydrocarbon radicals in formula (XI) are linear hydrocarbon radicals.

Examples of compounds of formula (XI) include, but are not limited to, diacyloxyethyidimethylammonium, diacyloxyethylhydroxyethylmethylammonium, monoacyloxyethyidihydroxyethylmethylammonium, triacyloxyethylmethylammonium, and monoacyloxyethylhydroxyethyidimethylammonium salts (for example, chloride and methyl sulphate), and mixtures thereof. The acyl radicals may comprise from 14 to 18 carbon atoms and may be obtained, for example, from a plant oil such as palm oil and sunflower oil. When the compound contains two or more acyl radicals, these radicals may be identical or different.

These products may be obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification may be followed by a quaternization using an alkylating agent such as an alkyl halide (for example, methyl and ethyl halide), a dialkyl sulphate (for example, dimethyl and diethyl sulphate), methyl methanesulphonate, methyl para-toluenesulphonate, glycol chlorohydrin, and glycerol chlorohydrin. Such compounds are sold, for example, under the names Dehyquart® by Henkel, Stepanquat® by Stepan, Noxamium® by CECA or Rewoquat® WE 18 by Rewo-Witco.

In at least one embodiment, the composition according to the present disclosure may comprise a mixture of quaternary ammonium mono-, di-, and triester salts, with a majority by weight of diester salts.

Non-limiting examples of mixture of ammonium salts include mixtures comprising from 15% to 30% by weight of acyloxyethyl-dihydroxyethyl-methylammonium methyl sulphate, from 45% to 60% of diacyloxyethyl-hydroxyethyl-methylammonium methyl sulphate, and from 15% to 30% of triacyloxyethyl-methylammonium methyl sulphate, wherein the acyl radicals may comprise from 14 to 18 carbon atoms and may originate from optionally partially hydrogenated palm oil.

Ammonium salts comprising at least one ester function, such as those described in U.S. Pat. Nos. 4,874,554 and 4,137,180 may also be used in accordance with the present disclosure.

In one embodiment, the quaternary ammonium salts of formula (VIII), may be chosen from tetraalkylammonium chlorides, for example, dialkyldimethylammonium chlorides and alkyltrimethylammonium chlorides, in which the alkyl radical comprises from 12 to 22 carbon atoms, for example, behenyltrimethylammonium chloride, distearyldimethylammonium chloride, cetyltrimethylammonium chloride, and benzyldimethylstearylammonium chloride; and palmitylamidopropyltrimethylammonium chloride and stearamidopropyldimethyl(myristyl acetate)ammonium chloride sold, for example, under the name Ceraphyl® 70 by Van Dyk.

In another embodiment of the present disclosure, the cationic surfactant may be chosen from quaternary ammonium salts, for example, behenyltrimethylammonium chloride and palmitylamidopropyltrimethylammonium chloride.

The at least one surfactant may be present in the composition in an amount ranging from 0.1% to 10% by weight, for example, from 0.5% to 8% by weight, or from 1% to 5% by weight relative to the total weight of the composition.

In at least one embodiment, the surfactant is chosen from amphoteric and nonionic surfactants.

The skilled person will ensure that any additives and their amount are chosen such that they are not detrimental to the properties of the compositions according to the present disclosure. These various additives may be present in the composition according to the present disclosure in an amount ranging from 0 to 20% by weight relative to the total weight of the composition.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below. The percentages are expressed on a weight basis of active material, unless otherwise indicated.

### EXAMPLES

### Example 1

### Lacquer

The following lacquer was prepared in accordance with the present disclosure:

| | |
|---|---|
| a. Avalure UR 450 (Noveon) | 1% a.s. |
| b. Eastman AQ 55S | 1% a.s. |
| c. Q2-5220 (Dow Corning) | 0.5% a.s. |
| d. water | 53.5% a.s. |
| e. DME | 40% a.s. |

### Example 2

### Aerosol Spray

The following water/air-type fixative spray fluid was prepared in accordance with the present disclosure and placed in a container under pressure:

| | |
|---|---|
| a. Avalure UR 450 (Noveon) | 1% a.s. |
| b. Eastman AQ 55S | 1% a.s. |
| c. water | 98% a.s. |

### Example 3

### Mousse

The following mousse was prepared in accordance with the present disclosure:

| | |
|---|---|
| a. Avalure UR 450 (Noveon) | 3% a.s. |
| b. oxyethylenated (20EO) sorbitan monolaurate | 0.2% a.s. |
| c. water | 88.8% a.s. |
| d. isobutane/propane (85/15) | 5% a.s. |
| e. Eastman AQ 55S | 3% a.s. |

### Example 4

### Styling Gel

The following styling gel was prepared in accordance with the present disclosure:

| | | |
|---|---|---|
| a. Avalure UR 450 (Noveon) | 5% | a.s. |
| b. Eastman AQ 55S | 5% | a.s. |
| c. Jaguar HP105 (Rhodia) | 1% | a.s. |
| d. Q2-5220 (Dow Corning) | 1% | a.s. |
| e. water | qs 100% | a.s. |

### Example 5

### Non-Aerosol Spray

The following non-aerosol fixative spray was prepared in accordance with the present invention:

| | |
|---|---|
| a. Avalure UR 450 (Noveon) | 3% a.s. |
| b. Eastman AQ 55S | 3% a.s. |
| c. water | 94% a.s. |

Eastman AQ 55S, sold by Eastman, is a diethylene glycol/1,4-cyclohexanedimethanol/isophthalate/sulphoisophthalate copolymer, which is a water-dispersible linear sulphonic polyester.

### Example 6

### Shape Retention Measurement

Operating Protocol:

2g of formula prepared in Example 5 was applied to a 2.7 g tress of natural hair, 27 cm long. The tress was wound around a roller 1 cm in diameter in order to shape it. The product was allowed to dry in air, then the tress was very carefully removed from the roller. The tresses thus shaped were subsequently immersed in a saltwater bath (3% NaCl) with a volume of 8 litres at room temperature, with magnetic stirring at 100 revolutions/minute. The length of the tress was measured over time in order to evaluate the shape retention.

Shape Retention Measurements:

Shape retention in %=(Lᵢ-L)/(Lᵢ-L₀)*100
L: length of the curled tress at time t
L₀: length of the curled tress after shaping and removal of the roller
Lᵢ: length of the tress before shaping on the roller

Results:

| Immersion time | Shape retention in % |
|---|---|
| 0 | 100 |
| 35 seconds | 100 |
| 40 seconds | 100 |
| 10 minutes | 100 |

What is claimed is:

1. A cosmetic composition comprising, in a cosmetically acceptable medium, at least one water-dispersible sulphonic polyester and at least one polyurethane having a number-average molecular weight ranging from 400 000 to 5 000 000 g/mol.

2. The composition of claim 1, wherein the at least one water-dispersible sulphonic polyester is linear.

3. The composition of claim 1, wherein the at least one water-dispersible sulphonic polyester is a polycondensate of at least one dicarboxylic acid or one of its esters, at least one diol, and at least one sulphoaryldicarboxylic difunctional compound substituted on the aromatic nucleus by a group —SO₃M, wherein M is chosen from hydrogen and metal ions.

4. The composition of claim 3, wherein the metal ions are chosen from Na⁺, Li⁺, and K⁺.

5. The composition of claim 1, wherein the at least one water-dispersible sulphonic polyester is obtained from isophthalic acid, the sodium salt of sulphoisophthalic acid, diethylene glycol, and 1,4-cyclohexanedimethanol.

6. The composition of claim 1, wherein the Tg of the at least one water-dispersible sulphonic polyester is greater than or equal to 50° C.

7. The composition of claim 1, wherein the at least one water-dispersible sulphonic polyester is branched.

8. The composition of claim 1, wherein the at least one water-dispersible sulphonic polyester is present in the composition in an amount ranging from 0.1% to 40% by weight relative to the total weight of the composition.

9. The composition of claim 8, wherein the at least one water-dispersible sulphonic polyester is present in the composition in an amount ranging from 1% to 30% by weight relative to the total weight of the composition.

10. The composition of claim 9, wherein the at least one water-dispersible sulphonic polyester is present in the composition in an amount ranging from 5% to 25% by weight relative to the total weight of the composition.

11. The composition of claim 1, wherein the at least one polyurethane is present in the composition in an amount ranging from 0.01% to 40% by weight relative to the total weight of the composition.

12. The composition of claim 11, wherein the at least one polyurethane is present in the composition in an amount ranging from 0.05% to 20% by weight relative to the total weight of the composition.

13. The composition of claim 1, wherein the at least one polyurethane is present in the composition in an amount ranging from 0.1% to 10% by weight relative to the total weight of the composition.

14. The composition of claim 1, further comprising at least one additional fixative polymer chosen from anionic, cationic, amphoteric, and nonionic fixative polymers, and mixtures thereof.

15. The composition of claim 14, wherein the cationic fixative polymer is chosen from homopolymers and copolymers of acrylic and methacrylic esters and amides comprising amino functions, cationic polysaccharides, quaternary vinylpyrrolidone-vinylimidazole copolymers, and chitosans.

16. The composition of claim 14, wherein the anionic fixative polymer is chosen from homopolymers and copolymers of acrylic and methacrylic acid and their salts, copolymers of crotonic acid, copolymers of C₄-C₈ monounsaturated carboxylic acids and anhyrides, polyacrylamides comprising carboxylate groups, homopolymers and copolymers comprising sulphonic groups, anionic polyurethanes, and grafted anionic silicone polymers.

17. The composition of claim 14, wherein the amphoteric fixative polymer is chosen from copolymers comprising acidic vinyl units and basic vinyl units, crosslinked and acylated polyaminoamides, polymers comprising zwitterionic units, polymers derived from chitosan, modified (C₁-C₅)alkyl vinyl ether/maleic anhydride copolymers, amphoteric polyurethanes, and grafted amphoteric silicone polymers.

18. The composition of claim 14, wherein the nonionic fixative polymer is chosen from polyalkyloxazolines, vinyl acetate homopolymers and copolymers, acrylic ester homopolymers and copolymers, acrylonitrile copolymers, styrene homopolymers and copolymers, polyamides, vinyllactam homopolymers other than vinylpyrrolidone homopolymers, vinyllactam copolymers, nonionic polyurethanes, and grafted nonionic silicone polymers.

19. The composition of claim 14, wherein the at least one additional fixative polymer is present in the composition in an amount ranging from 0.1% to 20%by weight relative to the total weight of the composition.

20. The composition of claim 19, wherein the at least one additional fixative polymer is present in the composition in an amount ranging from 0.5% to 1I0%by weight relative to the total weight of the composition.

21. The composition of claim 1, wherein the cosmetically acceptable medium comprises water or a mixture of water and at least one organic solvent.

22. The compsition of claim 21, wherein said at least one organic solvent is chosen from C₁-C₄ lower alcohols.

23. The composition of claim 22, wherein the C₁-C₄ lower alcohols are chosen from ethanol, isopropanol, tert-butanol, n-butanol, polyols, and polyol ethers.

24. The composition of claim 1, further comprising at least one compound chosen from silicones, silicone fatty substances, and non-silicone fatty substances.

25. The composition of claim 24, wherein the at least one compound chosen from silicones, silicone fatty substances, and non-silicone fatty substances is present in the composition in an amount ranging from 0.01% to 20% by weight relative to the total weight of the composition.

26. The composition of claim 25, wherein the at least one compound chosen from silicones, silicone fatty substances, and non-silicone fatty substances is present in the composition in an amount ranging from 0.05% to 10% by weight relative to the total weight of the composition.

27. The composition of claim 1, further comprising at least one additive chosen from nonionic, anionic, cationic, and amphoteric surfactants; additional nonionic, anionic, cationic, and amphoteric polymers; ceramides and pseudoceramides; vitamins and provitamins; silicone and non-silicone, water-soluble and lipid-soluble sunscreens; fillers and solid particles, nacreous agents and opacifiers; flakes; active particles; dyes; sequestrants; plasticizers; solubilizers; acidifying agents; alkalifying agents; neutralizing agents; organic and inorganic thickeners; antioxidants; hydroxy acids; penetrants; fragrances; and preservatives.

28. The composition of claim 27, wherein said fillers and said solid particles are chosen from colored and uncolored mineral and organic pigments.

29. The composition of claim 1, wherein the composition does not comprise C₁-C₄ alcohols.

30. A method for shaping, fixing, and/or holding a hairstyle, comprising applying a cosmetic composition to the hair, wherein the cosmetic composition comprises, in a cosmetically acceptable medium, at least one water-dispersible sulphonic polyester and at least one polyurethane having a number-average molecular weight ranging from 400 000 to 5 000 000 g/mol.

31. The method of claim 30, wherein the hairstyle is water-resistant.
